(19) United States
(12) Patent Application Publication
(10) Pub. No.: US 2024/0382409 A1
(43) Pub. Date: Nov. 21, 2024

(54) COMPOSITION FOR ANTI-AGING AND SKIN IMPROVEMENT, COMPRISING COMPLEX OF INDIAN GOOSE-BERRY EXTRACT AND BARLEY SPROUT EXTRACT (lB COMPLEX) AS ACTIVE INGREDIENT

(71) Applicant: HLSCIENCE CO., LTD, Gyeonggi-do (KR)

(72) Inventors: Hae-Yeon LEE, Gyeonggi-do (KR); Jong-Lae KIM, Gyeonggi-do (KR); Sung Bin IM, Gyeonggi-do (KR); So Young KIM, Gyeonggi-do (KR)

(21) Appl. No.: 18/693,818

(22) PCT Filed: Sep. 29, 2022
(86) PCT No.: PCT/KR2022/014704

§ 371 Date: Mar. 20, 2024

(30) Foreign Application Priority Data
Oct. 6, 2021 (KR) 10-2021-0132678

(51) Int. Cl. A61K 8/9789; A61K 8/9794; A61K 36/185; A61K 36/899; A61Q 19/00; A61Q 19/08

(52) U.S. Cl. CPC A61K 8/9789; A61K 8/9794; A61K 36/185; A61K 36/899; A61Q 19/007; A61Q 19/08; A61K 2236/31; A61K 2236/331; A61K 2236/51; A61K 2800/74; A61K 2800/805

(57) ABSTRACT

The present invention relates to a health functional food, a food and a cosmetic composition for anti-oxidation, wrinkle improvement, elasticity improvement and skin moisturizing comprising a complex of an Indian gooseberry (Phyllanthus emblica) extract and a barley sprout (Hordeum vulgare) as an active ingredient. The complex provided from the present invention has excellent DPPH scavenging activity, elastase inhibitory activity, MMP-1 inhibitory activity and hyaluronic acid content increasing effect, so it can be used as a health functional food, a food and a cosmetic composition for anti-oxidation, wrinkle improvement, elasticity improvement and skin moisturizing.

### TECHNICAL FIELD

The present application claims the priority based on Korean Patent Application No. 10-2021-0132678 filed on Oct. 6, 2021, and the entire contents disclosed in the description and drawings of the corresponding application are incorporated in the present application.

The present invention relates to a composition for anti-aging and skin improvement comprising natural extracts as an active ingredient.

### BACKGROUND ART

Active oxygen flowing in from outside a living body or generated in a living body accelerates aging of the living body or causes cancer. Therefore, research on anti-oxidant substances that inhibit oxidation by active oxygen is in progress, and the anti-oxidant substances such as phenolic compounds, flavonoids, tocopherol, vitamin C, selenium and the like are known. However, the anti-oxidant substance present in nature cannot be expected to have a practically sufficient effect when applied to skin. On the other hand, some inexpensive synthetic anti-oxidant agents with high anti-oxidation power have a problem in that their use is limited due to safety concerns such as human body side effects and the like.

Skin is the first organ to function as a physical barrier to protect a body against various environmental factors, and various environmental stimulus cause activation of the immune system. Skin aging can be divided intrinsic aging which occurs due to a decrease in the physiological function and a structural change of skin with aging, and extrinsic aging which is caused by chemical stress due to external stimuli such as ultraviolet rays. Intrinsic aging is caused by reactive oxygen species (ROS) and the like generated during the metabolic process of cells present in skin, and extrinsic aging is caused by external factors such as UV and pollution, and photoaging accounts for most of extrinsic aging. Common phenomena found in skin changes caused by intrinsic and extrinsic aging are loss of skin elasticity due to reduction and modification of collagen and elastin which are main constituent proteins of the dermis and reduction of hyaluronic acid which is a matrix of the dermis. The dermis is composed of a fiber component and a matrix component, and as the fiber component, collagen plays a role of giving intensity and tension to skin and protecting skin, and accounts for 90% of the dermal layer. Elastin accounts for about 3˜4% of the dermal layer and affects the elasticity of skin. In particular, according to the research results so far, it is known that reduction and modification of collagen and elastin which are main proteins in the dermis are directly involved in generation of wrinkles and decrease of skin elasticity. In addition, the synthesis and degradation process of collagen in a human body is appropriately regulated, and as aging progresses, the activity of matrix metalloproteinases (MMPs), which are matrix proteolytic enzymes in skin, increases, which causes reduction of elasticity and wrinkle generation through a decrease in a collagen content of skin.

Hyaluronic acid is a component that maintains moisture and elasticity of skin, and is mainly synthesized by epidermal keratinocytes and dermal fibroblasts, and is a substance which plays an important role in creating an environment in which cells can function normally by binding to water. Reduction of hyaluronic acid content in skin is a major cause of skin aging by reducing skin elasticity and reducing the moisture content.

As such, studies on a skin improvement effect to prevent aging are being actively conducted in various fields. In particular, researches using natural substances are continued in order not to cause toxicity or irritation to a human body.

### DISCLOSURE

### Technical Problem

An object of the present invention is to provide a health functional food, a food and a cosmetic composition capable of showing skin aging prevention and improvement effects by giving effects of anti-oxidation, wrinkle improvement, elasticity improvement and skin moisturizing at the same time.

Another object of the present invention is to provide a health functional food, a food and a cosmetic comprising the composition.

Other object of the present invention is to provide a composition for anti-aging and skin improvement comprising a complex of an Indian gooseberry extract and a barley sprout extract as an active ingredient. The composition may have one or more uses selected from the group consisting of skin anti-oxidation, wrinkle improvement, elasticity improvement and skin moisturizing.

More specifically, an object of the present invention is to provide the following embodiments.

Embodiment 1. A composition for anti-oxidation, wrinkle improvement, elasticity improvement and/or skin moisturizing comprising a complex of an Indian gooseberry (Phyllanthus emblica) extract and a barley sprout (Hordeum vulgare) extract as an active ingredient; a use of a complex of an Indian gooseberry (Phyllanthus emblica) extract and a barley sprout (Hordeum vulgare) extract, for anti-oxidation, wrinkle improvement, elasticity improvement and/or skin moisturizing, preferably, for skin anti-oxidation, skin wrinkle improvement, skin elasticity improvement and/or skin moisturizing; a use of a complex of an Indian gooseberry (Phyllanthus emblica) extract and a barley sprout (Hordeum vulgare) extract, in the manufacture of a food product for anti-oxidation, wrinkle improvement, elasticity improvement and/or skin moisturizing; or a use of a complex of an Indian gooseberry (Phyllanthus emblica) extract and a barley sprout (Hordeum vulgare) extract, in the manufacture of a cosmetic product for anti-oxidation, wrinkle improvement, elasticity improvement and/or skin moisturizing.

Embodiment 2. The composition or use according to Embodiment 1, wherein the Indian gooseberry extract or barley sprout extract is juiced or extracted with a solvent selected from the group consisting of water, low carbon alcohols having 1 to 4 carbon atoms and mixed solvents thereof.

Embodiment 3. The composition or use according to any one of the preceding embodiments, wherein the complex of the Indian gooseberry extract and barley sprout extract is obtained by juicing raw materials.

Embodiment 4. The composition or use according to any one of the preceding embodiments, wherein the Indian gooseberry extract or barley sprout extract is a dry powder.

Embodiment 5. The composition or use according to any one of the preceding embodiments, wherein in the complex of the Indian gooseberry extract and barley sprout extract, the Indian gooseberry extract is mixed with the barley sprout extract at a weight ratio of 1:1 to 4:1 (Indian gooseberry extract: barley sprout extract).

Embodiment 6. The composition or use according to any one of the preceding embodiments, wherein the complex of the Indian gooseberry extract and barley sprout extract has DPPH scavenging activity, elastase inhibition activity, MMP-1 inhibition activity and/or a hyaluronic acid content increasing effect.

Embodiment 7. A health functional food product comprising the composition described in any one of the preceding embodiments.

Embodiment 8. A food product comprising the composition described in any one of the preceding embodiments.

Embodiment 9. A cosmetic composition for anti-oxidation, wrinkle improvement, elasticity improvement and/or skin moisturizing comprising the complex of an Indian gooseberry (Phyllanthus emblica) extract and a barley sprout (Hordeum vulgare) extract described in any one of the preceding embodiments.

Embodiment 10. The composition or use according to any one of the preceding embodiments, wherein the Indian gooseberry extract or barley sprout extract is juiced or extracted with a solvent selected from the group consisting of water, low carbon alcohols having 1 to 4 carbon atoms and mixed solvents thereof.

Embodiment 11. The composition or use according to any one of the preceding embodiments, wherein the complex of the Indian gooseberry extract and barley sprout extract is obtained by juicing raw materials.

Embodiment 12. The composition or use according to any one of the preceding embodiments, wherein the Indian gooseberry extract or barley sprout extract is a dry powder.

Embodiment 13. The composition or use according to any one of the preceding embodiments, wherein in the complex of the Indian gooseberry extract and barley sprout extract, the Indian gooseberry extract is mixed with the barley sprout extract at a weight ratio of 1:1 to 4:1.

Embodiment 14. The composition or use according to any one of the preceding embodiments, wherein the complex of the Indian gooseberry extract and barley sprout extract has DPPH scavenging activity, elastase inhibition activity, MMP-1 inhibition activity and/or a hyaluronic acid increasing effect.

Embodiment 15. A cosmetic product comprising the composition described in any one of the preceding embodiments.

Embodiment 16. A method for anti-oxidation, wrinkle improvement, elasticity improvement and/or skin moisturizing, comprising administering the composition described in any one of the preceding embodiments to a subject in need thereof.

Embodiment 17. A method for manufacturing of the composition described in any one of the preceding embodiments, comprising
obtaining an Indian gooseberry extract from Indian gooseberry fruits by comprising juicing and drying processes;
obtaining a barley sprout extract from barley sprout by comprising juicing and drying processes; and
mixing the Indian gooseberry extract and barley sprout extract at a weight ratio of 4:1 to 1:1 to prepare a complex.

Other objects and advantages of the present invention will become more apparent from the following detailed description of the invention, claims and drawings.

### Technical Solution

In order to achieve the above objects, the present invention provides a health functional food, a food and a cosmetic composition for anti-aging and skin improvement comprising a complex of an Indian gooseberry (Phyllanthus emblica) extract and a barley sprout (Hordeum vulgare) extract as an active ingredient.

Indian gooseberry (Phyllanthus emblica L.) is a plant also called Emblica officinalis Gaertn or Amla and distributed and growing throughout the wide area such as Southern Asia including Nepal and Southeast Asia including Malaysia region and central and southern regions in the wild. As arable land, it is widely cultivated in the slopes of mountains above 1500 m above sea level in Taiwan and in the Himalayas. Indian gooseberry is a deciduous tree with a height of 3˜8 m, and the length of its leaf is about 10 mm, and the width is rectangular of about 2˜3 mm, and small lemon-scented yellow green flowers bloom in April˜May. The fruit is a flat ball shape with an average size of 18˜25 mm, and when ripe, it is light yellow and shiny and is yellow-green in six pieces of lines. The taste of fresh fruit is sweet, sour, and bitter and aftertaste is sweet. The fruit turns black when dried and the nutrients do not weaken even when dried, so dried ones are distributed. Amla is also called amalaki, which is considered a sacred tree in Nepal. In the Indian gooseberry (amla) is known to contain vitamin C, minerals, amino acids, tannins, rutin, and the like. Vitamin C of the Indian gooseberry is contained 20 times more than oranges, and is very stable to heat, so even prolonged exposure to a high temperature hardly destroys the vitamin. It is believed that the heat resistance of vitamin C comes from the tannin component included together, and these components show anti-oxidation, anti-tumor and anti-inflammatory activities, and the like. In addition, in the Indian gooseberry, various bioactive compounds such as ellagic acid, gallic acid, quercetin and the like are comprised.

Barley sprout (Hordeum vulgare L.) refers to the state of young leaves grown by sowing barley seeds with young shoots of barley. In one embodiment, the barley sprout may be a young leaf of about 10˜20 cm in about 8˜15 days after sowing. Barley is one of the first crops cultivated on the Eurasian continent about 10,000 years ago, and is known as one of the oldest crops cultivated by mankind. The barley sprout is known as a nutritionally excellent food source as it not only has a high content of various vitamins including vitamins A, B and vitamin C, and minerals such as calcium, magnesium and potassium and the like, but also contains a large amount of dietary fiber, so the potential for industrial application as functional food and medicine materials is increasing. In the United States, Japan and the like, young barley leaves are freeze-dried and powdered, which is developed and sold as a health food, and in Korea, as a general food and a health functional food, it is variously marketed and sold as powder, pills, green juice, tea, cosmetics and the like. In terms of pharmacological activity, the barley sprout exhibits various functions, such as anti-oxidation activity, smooth bowel movements, cholesterol level improvement, blood glucose level improvement, hyperlipidemia improvement and anti-obesity effects and the like by various bioactive compounds. These biological activities are believed to originate from flavonoids including saponarin, lutonarin and the like.

Accordingly, the present inventors have found the fact that the complex of an Indian gooseberry extract and a barley sprout extract shows excellent DPPH scavenging activity, elastase inhibition activity, MMP-1 inhibition activity and a hyaluronic acid content increasing effect at the same time, and gives effects of anti-oxidation, wrinkle improvement, elasticity improvement and skin moisturizing simultaneously, and therefore, can exhibit skin aging prevention and improvement effects, thereby completing the present invention.

In the present invention, the complex of an Indian gooseberry extract and a barley sprout extract is characterized by being juiced or extracted with a solvent selected from the group consisting of water, low carbon alcohols having 1 to 4 carbon atoms and mixed solvents thereof.

In the present invention, the complex of an Indian gooseberry extract and a barley sprout extract is characterized by being obtained by juicing raw materials.

In the present invention, the complex of an Indian gooseberry extract and a barley sprout extract is characterized by being a dry powder.

In the present invention, the complex of an Indian gooseberry extract and a barley sprout extract is characterized in that the Indian gooseberry extract is mixed at a weight ratio of 1:1 to 4:1 compared to the barley sprout extract.

In the present invention, the complex of an Indian gooseberry extract and a barley sprout extract is characterized by having DPPH scavenging activity, elastase inhibition activity, MMP-1 inhibition activity and a hyaluronic acid content increasing effect.

In addition, the present invention provides a health functional food, a food and a cosmetic comprising the composition.

### Advantageous Effects

The complex provided from the present invention has excellent DPPH scavenging activity, elastase inhibitory activity, MMP-1 inhibitory activity and hyaluronic acid content increasing effect, so it can be used as a health functional food, a food and a cosmetic composition for anti-oxidation, wrinkle improvement, elasticity improvement and skin moisturizing.

### BRIEF DESCRIPTION OF THE DRAWINGS

FIG. 1 is a graph showing the evaluation of free radical scavenging activity of Example 5.

FIG. 2 is a graph showing the elastase inhibition activity of Example 5.

FIG. 3 is a graph showing the MMP-1 inhibition activity of Example 5.

### MODE FOR INVENTION

Unless defined otherwise, all technical and scientific terms used in the present description have the same meaning as commonly understood by those skilled in the art to which the present invention pertains. In general, the nomenclature used in the present description is well known and commonly used in the present technical field.

Throughout the present description, when a part “comprises” any component, this means that other components may be further comprised, rather than excluding other components, unless otherwise described.

According to one embodiment of the present invention, the present invention relates to a health functional food, a food and a cosmetic composition for anti-aging and skin improvement comprising a complex of an Indian gooseberry (Phyllanthus emblica) extract and a barley sprout (Hordeum vulgare) extract as an active ingredient.

The extract according to the present invention may be obtained by various extraction methods known in the art to which the present invention pertains, and specifically, it may be obtained by juicing raw materials or extracted with a solvent selected from the group consisting of water, organic solvents and mixed solvents thereof. Among them, juicing raw materials, or extracting with water or alcohol is more preferable in terms of stably extracting an active ingredient.

The water may be alkaline water, and the organic solvent may include one or more kinds of polar organic solvents such as anhydrous or hydrous low carbon alcohols having 1 to 4 carbon atoms, for example, methanol, ethanol, propanol, n-butanol, acetone and the like; non-polar organic solvents such as ether, hexane, benzene, chloroform, ethyl acetate, and the like; vegetable organic solvents such as soybean oil, sesame oil, and the like; and mixtures thereof. The organic solvent may be used alone or as a mixture of two or more kinds.

As the solvent used during extraction, one or more organic solvents among methanol, ethanol, propanol and n-butanol and a mixed solvent by comprising water in these solvents may be used. When the mixed solvent is used as the extraction solvent, the extraction efficiency of the active ingredient can be further improved. Then, when the mixed solvent is used, it may be appropriately mixed and used according to any purpose.

In addition, the extract may be extracted at room temperature or by heating under the condition in which the active ingredient of the extract is not destroyed or destruction of the active ingredient is minimized. The extraction method is not particularly limited and according to the kind and extracting method of the extract, for example, juicing, water bath extraction, reflux cooling extraction, ultrasonic extraction, and enfleurage extraction methods may be used.

In the extracting step, the extraction time may be specifically repeated 1 to 5 times. In this case, the extraction efficiency of the active ingredient may be further improved.

As one example, the extracting step may be extracted by juicing the extraction materials by applying pressure at room temperature, without adding extraction solvent.

As another method, the extracting step may be extracted by heating in boiling water at 50 to 100° C. for 0.5 to 72 hours, after adding extraction solvent to extraction materials.

As other method, the extracting step may perform reflux cooling extraction at 50 to 100° C. for 4 to 20 hours, after adding extraction materials and extraction solvent into an extractor equipped with a reflux cooler. Then, the reflux cooling extraction is a method of extracting by cooling after reflux while heating, and specifically, means an extraction method which minimizes solvent loss, by conducting a cooling process together as evaporation may occur due to heating and therefore, solvent loss may occur.

As other method, the extracting step may be extracted by depositing and enfleurage at 5 to 37° C. for 1 to 15 days, after adding extraction solvent to extraction materials.

As other method, the extracting step may obtain extraction liquid in a concentrate state by adding ethanol of 10 to 50 folds of the extraction materials weight based on the dry solid of the plant complex, extracting at 20 to 80° C. for 1 to 72 hours, filtering, and concentrating.

Furthermore, the extraction materials of the present invention include not only extraction materials extracted by the aforementioned extraction solvent, but also extraction materials extracted through a common purification process. For example, fractions obtained by passing through an ultrafiltration membrane having a constant molecular weight cut-off value, and active fractions obtained through various purification methods performed additionally, such as separation by various chromatography (prepared for separation according to size, charge, hydrophobicity or hydrophilicity), and the like are included in the extract of the present invention.

Filtration is a process of removing suspended solid particles from the extraction liquid, and may filter particles using cotton, nylon, paper, and the like, or use ultrafiltration, cryofiltration, centrifugation and the like, but not limited thereto.

The step of drying after concentrating the filtrate may include freeze drying, vacuum drying, hot air drying, spray drying, decompression drying, foam mat drying, high frequency drying, infrared drying, and the like, but not limited thereto. In some cases, a process of pulverizing the final dried extract may be added.

According to one embodiment of the present invention, the extract may be used as processed in various forms such as an extract, powder obtained by drying the extract, concentrate obtained by concentrating and filtering the extract, and the like. In addition, the extract is not particularly limited, but for example, it may be used in a form of tincture, concentrate, extract, fluid extract and the like, which comprise hydrous alcohol as a leaching solvent.

According to another embodiment of the present invention, the Indian gooseberry extract prepared above may comprise ellagic acid of 1 to 25 mg/g, and may comprise ellagic acid of 1 to 5 mg/g under the condition of acid hydrolysis according to the analysis method, and may comprise free ellagic acid of 5 to 25 mg/g under the condition without acid hydrolysis. In addition, the barley sprout extract prepared above may comprise saponarin of 6 to 11 mg/g.

According to other embodiment of the present invention, the complex of the Indian gooseberry extract and barley sprout extract is preferably made by mixing the Indian gooseberry extract and barley sprout extract at a weight ratio of 1:1 to 4:1 (Indian gooseberry extract: barley sprout extract), and it is the most preferable that it is made by mixing them at a weight ratio of 1:1 to 2:1 (Indian gooseberry extract: barley sprout extract) in the aspect of being able to improve all the effects of anti-oxidation, wrinkle improvement, elasticity improvement and skin moisturizing. If it is out of the above range, it is not preferable because the effects of anti-oxidation, wrinkle improvement, elasticity improvement and skin moisturizing cannot be improved at the same time.

The complex of Indian gooseberry extract and barley sprout extract can offer the DPPH scavenging activity, elastase inhibition activity, MMP-1 inhibition activity and hyaluronic acid content increasing effect at the same time, so it has not only simple skin care, but also an effect capable of implementing fundamental skin health improvement.

As aforementioned, the complex of Indian gooseberry extract and barley sprout extract has excellent effects of anti-oxidation, wrinkle improvement, elasticity improvement and skin moisturizing, so it can be used as a health functional food, a food and a cosmetic composition for anti-oxidation, wrinkle improvement, elasticity improvement and skin moisturizing.

According to one embodiment of the present invention, the food composition may be used as a food, a food additive, a beverage, a beverage additive, fermented milk, a health functional food, or the like. When used as a food, a food additive, a beverage, a beverage additive or a health functional food, it may be various kinds of foods, fermented milk, meat, beverages, chocolate, snacks, confectionery, pizza, ramen, other noodles, gum, ice cream, alcohol beverages, vitamin complexes, liquor and other health functional foods, but not limited thereto.

When the complex of the Indian gooseberry extract and barley sprout extract of the present invention is prepared as a food composition, it may comprise not only the Indian gooseberry and barley sprout as an active ingredient, but also components commonly added during food manufacturing.

The additional components include for example, proteins, carbohydrates, fats, nutrients, seasonings and flavoring agents. The carbohydrate is common saccharides such as monosaccharides (for example, glucose, fructose, etc.), disaccharides (for example, maltose, sucrose, oligosaccharide, etc.) and polysaccharides (for example, dextrin, cyclodextrin, etc.) and sugar alcohols such as xylitol, sorbitol, erythritol, and the like. As the flavoring agent, a natural flavoring agent (thaumatin, stevia extract (for example, rebaudioside A, glycyrrhizin, etc.)) and a synthetic flavoring agent (saccharin, aspartame, etc.) may be used.

For example, when the food composition of the present invention is manufactured as a beverage, pectin, citric acid, liquid fructose, sugar, glucose, acetic acid, malic acid, fruit juice and the like may be further comprised in addition to the active ingredient of the present invention, Indian gooseberry and barley sprout.

The food composition of the present invention includes processed forms of all natural materials such as food, functional food, nutritional supplement, health food and food additives, and the like. The food composition in these types may be manufactured in various forms according to a conventional method known in the art.

For example, as the health food, the complex of the Indian gooseberry extract and barley sprout extract may be manufactured and consumed in a form of tea, juice and drink, or may be ingested by granulation, encapsulation and powdering. In addition, as the food, it may be manufactured by adding the plants extract complex of the present invention such as beverages (including alcoholic beverages), fruits and processed foods thereof (for example, canned fruit, bottled fruit, jam, marmalade, etc.), fish, meat and processed foods thereof (for example, ham, sausage, corned beef, etc.), breads and noodles (for example, udon noodles, soba noodles, ramen, spaghetti, macaroni, etc.), fruit juice, various kinds of drinks, cookies, taffy, dairy products (for example, yogurt, fermented milk, butter, cheese, etc.), edible vegetable oil, margarine, vegetable protein, retort food, frozen food, various kinds of seasonings (for example, soybean paste, soy sauce, sauce, etc.) and the like. Moreover, in order to use the complex of the Indian gooseberry extract and barley sprout extract of the present invention in a form of a food additive, it may be prepared and used in a form of powder or concentrate.

According to one embodiment of the present invention, the usage of the food composition for anti-oxidation, wrinkle improvement, elasticity improvement and skin moisturizing may be appropriately adjusted according to an individual difference such as age, health condition, and the like, formulation and type, and the composition may be usefully used as a food composition for anti-oxidation, wrinkle improvement, elasticity improvement and skin moisturizing.

The composition obtained from the above method may provide a health functional food and a food excellent for anti-oxidation, wrinkle improvement, elasticity improvement and skin moisturizing by exhibiting the DPPH scavenging activity, elastase inhibition activity, MMP-1 inhibition activity and hyaluronic acid content increasing effect.

According to one embodiment of the present invention, the cosmetic composition may comprise one or more kinds selected from the group consisting of purified water, glycerin, myristic acid, butylene glycol, potassium hydroxide, polyethylene glycol, stearate, stearic acid, lauric acid, lauramide DEA, glyceryl stearate, behenyl alcohol, microcrystalline wax, phenoxyethanol, ethylhexylglycerin, tocopheryl acetate, 1,2-hexanediol, caprylyl glycol, hydroxyethylene cellulose, xanthan gum, collagen, sodium citrate, citrate, magnesium ascorbate and fragrance.

In addition, in the cosmetic composition of the present invention, other components mixed to common cosmetics may be mixed if needed. As one example, fat and oil components, moisturizers, emollient agents, surfactants, organic and inorganic pigments, organic powder, ultraviolet absorbers, preservatives, disinfectants, antioxidants, plant extract, pH adjusting agents, alcohols, pigments, fragrances, blood circulation accelerators, cooling agents, antiperspirants or purified water is preferable, but not limited thereto.

According to one embodiment of the present invention, when used as the cosmetic composition, it may have any one formulation selected from the group consisting of skin ointment, cream, skin, astringent, lotion, pack, mask sheet, essence, hair tonic, shampoo, rinse, hair conditioner, hair treatment, spray, paste, gel, gel ointment, patch, skin lotion, skin softener, skin toner, astringent lotion, milk lotion, moisture lotion, nutritional lotion, massage cream, nutritional cream, eye cream, moisture cream, hand cream, foundation, powder foundation, nutritional essence, sunscreen, soap, cleansing oil, cleansing foam, cleansing lotion, cleansing cream, cleansing water, powder, body lotion and body cleanser, but not limited thereto. The composition of each of these formulations may contain various bases and additives necessary and appropriate for formulation of the formulation, and the type and amount of these components may be easily selected by those skilled in the art.

When the formulation of the cosmetic composition of the present invention is paste, cream or gel, as a carrier component, animal oil, vegetable oil, wax, paraffin, starch, cellulose derivatives, polyethylene glycol, silicon, bentonite, silica, gum tragacanth, talc or zinc oxide, titanium oxide, or the like may be used.

When the formulation of the cosmetic composition of the present invention is body gel, in addition to the aforementioned active ingredients, carbomer, triethanolamine, menthol, ethanol, glycerin, disodium EDTA, methyl paraben, flavor and the like, which are components generally used during body gel manufacturing may be used. Herein, carbomer and triethanolamine acts as a thickener, and menthol acts as a flavor component, and ethanol functions to offer refreshment, and glycerin plays a role as a moisturizer, and disodium EDTA functions a metal component chelate, and methyl paraben is added for a function as a preservative. In addition, as the flavor, various flavors such as herb flavor and the like may be added in addition to the menthol flavor.

That a composition is prepared by applying known materials widely used in the present art in order to achieve functions of the aforementioned thickener, refreshment offering, flavor component, moisturizer and the like, in addition to the aforementioned specific components, may also be comprised in the scope of the present invention.

When the formulation of the present invention is powder or spray, as a carrier component, lactose, talc, silica, aluminum hydroxide, calcium silicate or polyamide powder may be added, and in particular, in case of spray, a propellant such as chlorofluorohydrocarbon, propane/butane or dimethylether may be additionally comprised.

When the formulation of the present invention is liquid or emulsion, as a carrier component, a solvent, a solubilizer, or an emulsifier is added, and the example thereof includes water, ethanol, isopropanol, ethyl carbonate, ethyl acetate, benzyl alcohol, benzyl benzoate, propylene glycol, 1,3-butylene glycol, glycerol aliphatic ester, polyethylene glycol, sorbitan fatty acid ester, and the like.

When the formulation of the present invention is suspension, as a carrier component, a liquid diluent such as water, ethanol or propylene glycol, suspension such as ethoxylated isostearyl alcohol and polyoxyethylene sorbitan ester, microcrystalline cellulose, aluminum hydroxide, bentonite, agar, gum tragacanth or the like may be used.

When the formulation of the present invention is cleansing containing surfactant, as a carrier component, aliphatic alcohol sulfate, aliphatic alcohol ether sulfate, sulfosuccinate monoester, isethionate, imidazolinium derivatives, methyl taurate, sarcosinate, fatty acid amide ether sulfate, alkylamido betaine, aliphatic alcohol, fatty acid glyceride, fatty acid diethanolamide, vegetable oil, lanolin derivatives or ethoxylated glycerol fatty acid ester, or the like may be used.

According to one embodiment of the present invention, the cosmetic composition for anti-oxidation, wrinkle improvement, elasticity improvement and skin moisturizing may be prepared as a composition such as body gel, body lotion, body cream, body oil and the like for promoting fat decomposition, and may be prepared in an aerosol type. In this case, the cosmetic composition is preferably used by a method of dermal administration which directly applies or sprays it onto skin.

According to one embodiment of the present invention, the usage of the cosmetic composition for anti-oxidation, wrinkle improvement, elasticity improvement and skin moisturizing may be appropriately adjusted according to an individual difference such as age, skin fat condition, formulation and type, and the composition may be usefully used as a composition for the effects of anti-oxidation, wrinkle improvement, elasticity improvement and skin moisturizing.

The composition obtained from the above method may provide a cosmetic excellent for anti-oxidation, wrinkle improvement, elasticity improvement and skin moisturizing by showing the DPPH scavenging activity, elastase inhibition activity, MMP-1 inhibition activity and hyaluronic acid content increasing effect.

Hereinafter, the present invention will be described in more detail by examples. These examples are intended to illustrate the present invention more specifically only, and it will be obvious to those skilled in the art to which the present invention pertains that the scope of the present invention is not limited by these examples according to the gist of the present invention.

### EXAMPLE

### I. Preparation of Test Materials and Analysis of Marker Compounds

### <Examples 1 to 7> Preparation of Complex of Indian Gooseberry Extract and Barley Sprout Extract

After washing and juicing fruits of Indian gooseberry, an Indian gooseberry fruit extract was obtained. After that, it was filtered and concentrated, and then dried to prepare an Indian gooseberry extract in a powder form. After harvesting and juicing barley sprouts, it was filtered to remove insoluble fibers such as cellulose and then dried. Then, the dried powder was homogenized to prepare a barley sprout extract. The prepared Indian gooseberry extract and barley sprout extract in a powder form were mixed at a weight ratio of Table 1 below to prepare a final complex of the Indian gooseberry extract and barley sprout extract.

TABLE 1

| | Mixing ratio of | Indian | |
|---|---|---|---|
| | Indian | gooseberry | barley sprout |
| | gooseberry:barley | extract | extract |
| Classification | sprout | (% by weight) | (% by weight) |
| Example 1 | 1:6 | 14.29 | 85.71 |
| Example 2 | 1:4 | 20 | 80 |
| Example 3 | 1:2 | 33.33 | 66.67 |
| Example 4 | 1:1 | 50 | 50 |
| Example 5 | 2:1 | 66.67 | 33.33 |
| Example 6 | 4:1 | 80 | 20 |
| Example 7 | 6:1 | 85.71 | 14.29 |

### <Comparative Example 1> Preparation of Indian Gooseberry Extract

After washing and juicing fruits of Indian gooseberry, an Indian gooseberry fruit extract was obtained. After that, it was filtered and concentrated, and then dried to prepare an Indian gooseberry extract in a powder form.

### <Comparative Example 2> Preparation of Barley Sprout Extract

After harvesting and juicing barley sprouts, it was filtered to remove insoluble fibers such as cellulose and then dried. After that, the dried powder was homogenized to prepare a barley sprout extract.

### Analysis of Marker Compounds

For analysis of marker compounds of the Indian gooseberry extract and barley sprout extract and standardization of raw material, 3 lots of samples were prepared for each raw material of the Indian gooseberry extract (Comparative example 1-1, Comparative example 1-2, Comparative example 1-3) and barley sprout extract (Comparative example 2-1, Comparative example 2-2, Comparative example 2-3).

The marker compound of the Indian gooseberry extract was set to ‘ellagic acid’, and the marker compound of the barley sprout extract was set to saponarin to analyze them. According to the analysis method, the content of the marker compound for the Indian gooseberry extract was summarized in Table 2 and Table 3, and the content of the marker compound of the barley sprout extract was summarized in Table 4.

More specifically, the analysis of the marker compound content of each extract was conducted as follows.

The analysis method used for quantification of ellagic acid of the Indian gooseberry extract was as follows.

The content of ellagic acid in the Indian gooseberry extract was measured using high performance liquid chromatography, after acid hydrolysis. For the analysis, a column of Capcellpak C18 UG120 (4.6 mm×50 mm, 5 82 m) was used to detect ellagic acid at a wavelength of 370 nm with a UV detector. The mobile phases were 6:4 mixture of 0.85% phosphoric acid solution and methanol (solvent A) and methanol (solvent B), and it was applied to the gradient elution method. As a result, The content of ellagic acid in the Indian gooseberry extract prepared according to Comparative example 1 above was analyzed to be in a range of 1˜5 mg/g.

TABLE 2

| | Content |
|---|---|
| | of ellagic |
| | acid |
| Classification | (mg/g) |
| Comparative example 1-1 | 3.17 |
| Comparative example 1-2 | 1.91 |
| Comparative example 1-3 | 4.65 |

In the analysis method used for quantification of free ellagic acid of the Indian gooseberry extract, the analysis method under the condition without acid hydrolysis was as follows.

The content of free ellagic acid in the Indian gooseberry extract was measured using high performance liquid chromatography after methanol ultrasonic extraction. For the analysis, a column of Capcellpak C18 UG120 (4.6 mm×50 mm, 5 μm) was used to detect ellagic acid at a wavelength of 370 nm with a UV detector. The mobile phases were 6:4 mixture of 0.85% phosphoric acid solution and methanol (solvent A) and methanol (solvent B), and it was applied to the gradient elution method. As a result, the content of free ellagic acid in the Indian gooseberry extract prepared according to Comparative example 1 above was analyzed to be in a range of 5˜25 mg/g.

TABLE 3

| | Content |
|---|---|
| | of free |
| | ellagic |
| | acid |
| Classification | (mg/g) |
| Comparative example 1-1 | 11.73 |
| Comparative example 1-2 | 5.36 |
| Comparative example 1-3 | 24.15 |

The analysis method used for quantification of saponarin of the barley sprout extract was as follows.

The content of saponarin in the barley sprout extract was measured using high performance liquid chromatography after methanol ultrasonic extraction. For the analysis, a column of Capcellpak C18 UG120 (4.6 mm×50 mm, 5 μm) was used to detect saponarin at a wavelength of 340 nm with a UV detector. The mobile phases were 0.1% formic acid (solvent A) and methanol (solvent B), and it was applied to the gradient elution method. As a result, the content of saponarin in the barley sprout extract prepared according to Comparative example 2 above was analyzed to be in a range of 6˜11 mg/g.

TABLE 4

| | Content of |
|---|---|
| | saponarin |
| Classification | (mg/g) |
| Comparative example 2-1 | 6.56 |
| Comparative example 2-2 | 7.38 |
| Comparative example 2-3 | 10.54 |

### II. Efficacy Evaluation

### <Experimental Example 1> Anti-Oxidation Test: DPPH Scavenging Activity

In order to investigate the anti-oxidant effect using Examples 1 to 7 and Comparative examples 1 to 2, the DPPH (2,2-diphenyl-1-picryl hydrazyl) radical scavenging activity was evaluated.

The Examples 1 to 7 and Comparative examples 1 to 2 were mixed with 55 μM DPPH solution 450 μl by concentration and then reacted at 4° C. for 30 minutes. After completing the reaction, each sample was placed in a 96-well plate and the absorbance of samples was measured at 520 nm using a micro-plate reader. Then, a sample untreated group was used as a control group, and the free radical scavenging activity (%) of the sample was calculated as Equation 1 below.

Then, as a positive control scavenging radicals, ascorbic acid was used, and the concentration of ascorbic acid when the free radical scavenging activity was 50% was shown as 1.63 μg/mL.

$$Free radical scavenging activity ( \% ) = \qquad [ Equation 1 ]$$

$$[ ( 1 - absorbance of sample - treated group /$$

$$absorbance of sample - untreated group ) \times 100 ]$$

TABLE 5

| | | DPPH |
|---|---|---|
| | | radical |
| | | scavenging |
| Sample | Treatment | activity |
| (mixing ratio) | concentration | (%) |
| Example 1 (1:6) | 5 μg/mL | 15.85 ± 2.18 |
| Example 2 (1:4) | 5 μg/mL | 20.17 ± 0.50 |
| Example 3 (1:2) | 5 μg/mL | 31.99 ± 1.50 |
| Example 4 (1:1) | 5 μg/mL | 53.89 ± 1.72 |
| Example 5 (2:1) | 5 μg/mL | 57.48 ± 1.80 |
| Example 6 (4:1) | 5 μg/mL | 56.62 ± 1.80 |
| Example 7 (6:1) | 5 μg/mL | 57.35 ± 1.96 |
| Comparative | 5 μg/mL | 41.83 ± 3.60 |
| example 1 (1:0) | | |
| Comparative | 5 μg/mL | 4.08 ± 2.26 |
| example 2 (0:1) | | |

| | |
|---|---|
| Ascorbic acid (IC₅₀) | 1.63 μg/mL |

IC₅₀ means the concentration of sample when the free radical scavenging activity of the treated sample is 50%. Table 5 above summarizes the free radical scavenging activity in 5 μg/mL of each sample. As a result, as shown in Table 5 above, Examples 4 to 7 showed the DPPH radical scavenging activity of 53.89% to 57.48% at the 5 μg/mL treatment concentration, and were significantly excellent compared to 4.08% to 41.83% at the 5 μg/mL treatment concentration of Comparative examples 1 to 2. It could be confirmed that the DPPH radical scavenging activity of Example 5 was the most excellent among them.

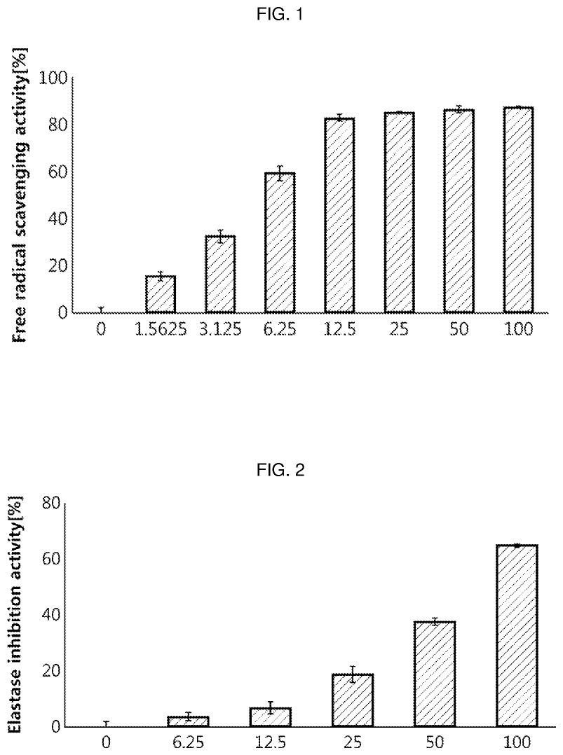

In addition, as shown in FIG. 1, when Example 5 was treated by concentration, the DPPH radical scavenging activity of Example 5 was increase in a dose-dependent manner, and it was confirmed that the anti-oxidant activity was very excellent.

### <Experimental Example 2> Efficacy Test of Wrinkle and Elasticity Improvement: Elastase Inhibition Activity

In order to investigate the improvement effect of wrinkle and elasticity using Examples 1 to 7 and Comparative examples 1 to 2, the elastase inhibition activity was evaluated.

The final concentration of the Examples 1 to 7 and Comparative examples 1 to 2 were prepared to be 100 μg/mL and a substrate at a concentration of N-succinyl-(Ala) 3-p-nitroanilide 1.6 mM dissolved in 10 mM Tris-HCL buffer (pH 8.0) and elastase from porcine pancreas Type IV 0.4 U/mL were mixed and reacted at 25° C. for 5 minutes, and then placed in a 96-well plate, and the absorbance of samples was measured at 410 nm using a micro-plate reader. Then, a sample untreated group was used as a control group, and the elastase inhibition activity (%) of the sample was calculated as Equation 2 below.

Then, it was used oleanolic acid as a positive control group, and the concentration of oleanolic acid when the elastase inhibition activity was 50% was shown as 19.63 μg/mL.

$$Elastase inhibition activity ( \% ) = \qquad [ Equation 2 ]$$

$$[ ( 1 - absorbance of sample treated group /$$

$$absorbance of sample untreated group ) \times 100 ]$$

TABLE 6

| | | Elastase |
|---|---|---|
| | | inhibition |
| Sample | Treatment | activity |
| (mixing ratio) | concentration | (%) |
| Example 1 (1:6) | 100 μg/mL | 75.28 ± 3.52 |
| Example 2 (1:4) | 100 μg/mL | 73.63 ± 2.14 |
| Example 3 (1:2) | 100 μg/mL | 71.76 ± 0.22 |
| Example 4 (1:1) | 100 μg/mL | 72.45 ± 1.14 |
| Example 5 (2:1) | 100 μg/mL | 70.89 ± 1.46 |
| Example 6 (4:1) | 100 μg/mL | 49.26 ± 0.87 |
| Example 7 (6:1) | 100 μg/mL | 39.78 ± 1.17 |
| Comparative | 100 μg/mL | 28.53 ± 0.50 |
| example 1 (1:0) | | |
| Comparative | 100 μg/mL | 45.52 ± 0.87 |
| example 2 (0:1) | | |

| | |
|---|---|
| Oleanolic acid (IC₅₀) | 19.63 μg/mL |

As a result, as shown in Table 6, it could be confirmed that the elastase inhibition activities shown at the 100 μg/mL treatment concentration of Examples 1 to 5 were significantly excellent as 70.89% to 75.28% compared to the elastase inhibition activity of 28.53% to 45.52% shown at the 100 μg/mL treatment concentration of Comparative examples 1 to 2.

In addition, as shown in FIG. 2, it was confirmed that the improving effects of wrinkle and elasticity were excellent, as the elastase inhibition activity was increased in a dose-dependent manner, when the complex of the Indian gooseberry extract and barley sprout extract of Example 5 was treated by concentration.

### <Experimental Example 3> Cytotoxicity Test: WST (Water-Soluble Tetrazolium Salt) Assay

In order to confirm toxicity to skin cells using Examples 1 to 7 and Comparative examples 1 to 2, a cytotoxicity experiment was performed in human keratinocyte HaCaT cells.

In order to confirm affecting toxicity to human keratinocyte HaCaT cells, cells were seeded at a concentration of 5×10⁵ cell/well in a 24-well plate using DMEM (Dulbecco's Modified Eagle Medium) medium, and then cultured under the condition of 37° C. and 5% CO₂ for 24 hours. Examples 1 to 7 and Comparative examples 1 to 2 were mixed with the medium so that the final concentration was 100 μg/mL to cells cultured for 24 hours, and treated to each well, and then cultured under the condition of 37° C. and 5% CO₂ for 24 hours. After 24 hours, the supernatant was removed and WST-1 assay solution was treated to each well, and reacted in an incubator for 2 hours, and then the absorbance was measured at 450 nm using a micro-plate reader, and the cell viability was calculated as Equation 3 below.

$$Cell viability ( \% ) = [ ( Absorbance of sample - treated group / \qquad [ Equation 3 ]$$

$$absorbance of sample - untreated group ) \times 100 ]$$

TABLE 7

| | | Cell |
|---|---|---|
| Sample | Treatment | viability |
| (mixing ratio) | concentration | (%) |
| Example 1 (1:6) | 100 μg/mL | 96.11 ± 2.56 |
| Example 2 (1:4) | 100 μg/mL | 96.39 ± 1.54 |
| Example 3 (1:2) | 100 μg/mL | 97.04 ± 1.01 |
| Example 4 (1:1) | 100 μg/mL | 97.19 ± 0.70 |
| Example 5 (2:1) | 100 μg/mL | 99.94 ± 1.83 |
| Example 6 (4:1) | 100 μg/mL | 96.16 ± 0.76 |
| Example 7 (6:1) | 100 μg/mL | 95.86 ± 0.66 |
| Comparative | 100 μg/mL | 95.53 ± 0.77 |
| example 1 (1:0) | | |
| Comparative | 100 μg/mL | 95.58 ± 1.06 |
| example 2 (0:1) | | |

As a result, as shown in Table 7 above, it was confirmed that the cytotoxicity was not shown at the 100 μg/mL treatment concentration of Examples 1 to 7 and Comparative examples 1 to 2, so it could be used as a safe material for foods and cosmetics, and in particular, it was confirmed that the cytotoxicity was not shown at all as the cell viability was 99.94% at a mixing ratio of Indian gooseberry extract and barley sprout extract 2:1 of Example 5.

### <Experimental Example 4> Wrinkle and Elasticity Improvement Test: MMP-1 (Matrix Metalloproteinase-1) Inhibition Activity

In order to investigate the improvement effect of wrinkle and elasticity using Examples 1 to 7 and Comparative examples 1 to 2 at the cellular level, the MMP-1 inhibition activity was evaluated in human keratinocyte HaCaT cells.

In order to evaluate the inhibition activity of MMP-1 which inducing wrinkle generation by degrading collagen in HaCaT cells, cells were seeded at a concentration of 5×10⁵ cell/well in a 24-well plate using DMEM (Dulbecco's Modified Eagle Medium) medium, and then cultured under the condition of 37° C. and 5% CO₂ for 24 hours. After simulating formation of MMP-1 by treating UV-B to the cells cultured for 24 hours, Examples 1 to 7 and Comparative examples 1 to 2 were mixed with the medium so that the final concentration was 100 μg/mL, and treated to each well, and then cultured under the condition of 37° C. and 5% CO₂ for 24 hours. After 24 hours, the supernatant was collected, and the MMP-1 concentration was measured according to the manual in the kit using MMP-1 ELISA kit (RAB0361), and the MMP-1 inhibition activity was calculated as Equation 4 below. The control group means a test group in which UV-B was treated but the sample was not treated.

$$MMP - 1 inhibition activity ( \% ) = [ \{ 1 - \qquad [ Equation 4 ]$$

$$( MMP - 1 concentration of UV - B - treated and sample - treated group /$$

$$MMP - 1 concentration of UV - B - treated and sample - untreated group ) \}$$

$$\times 100 ]$$

TABLE 8

| | | MMP-1 |
|---|---|---|
| | | inhibition |
| Sample | Treatment | activity |
| (mixing ratio) | concentration | (%) |
| Control group | 0 μg/mL | 0 ± 2.44 |
| Example 1 (1:6) | 100 μg/mL | 7.41 ± 0.69 |
| Example 2 (1:4) | 100 μg/mL | 9.79 ± 1.31 |
| Example 3 (1:2) | 100 μg/mL | 14.41 ± 0.16 |
| Example 4 (1:1) | 100 μg/mL | 23.33 ± 0.16 |
| Example 5 (2:1) | 100 μg/mL | 26.78 ± 0.06 |
| Example 6 (4:1) | 100 μg/mL | 17.92 ± 1.25 |
| Example 7 (6:1) | 100 μg/mL | 18.81 ± 2.12 |
| Comparative | 100 μg/mL | 13.55 ± 0.62 |
| example 1 (1:0) | | |
| Comparative | 100 μg/mL | 6.53 ± 0.16 |
| example 2 (0:1) | | |

As a result, as shown in Table 8, it could be confirmed that the MMP-1 inhibition activities of 23.33% to 26.78% shown at the 100 μg/mL treatment concentration of Examples 4 to 5 were significantly excellent compared to the MMP-1 inhibition activities of 13.55% to 6.53% shown at the 100 μg/mL treatment concentration of Comparative examples 1 to 2. It was confirmed that the MMP-1 inhibition effect of Example 5 was the most excellent among them.

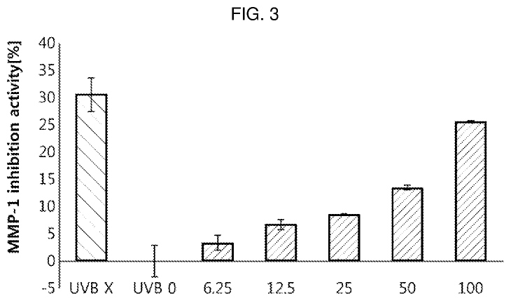

In addition, as shown in FIG. 3, it was confirmed that the improvement effect of wrinkle and elasticity was excellent, as the treatment of Example 5, complex of the Indian gooseberry extract and barley sprout extract, increased the MMP-1 inhibition activity in a dose-dependent manner.

### <Experimental Example 5> Skin Moisturizing Test: Hyaluronic Acid Content Measurement

In order to investigate the skin moisturizing effect using Examples 1 to 7 and Comparative examples 1 to 2 at the cellular level, the hyaluronic acid content was measured in human keratinocyte HaCaT cells.

In order to measure the content of hyaluronic acid showing a moisturizing effect by combining to water in HaCaT cells, cells were seeded at a concentration of 5×10⁵ cell/well in a 24-well plate using DMEM (Dulbecco's Modified Eagle Medium) medium, and then cultured under the condition of 37° C. and 5% CO₂ for 24 hours. Examples 1 to 7 and Comparative examples 1 to 2 were mixed with the medium so that the final concentration was 100 μg/mL to cells cultured for 24 hours, and treated to each well, and then cultured under the condition of 37° C. and 5% CO₂ for 24 hours. After 24 hours, the supernatant was collected, and the hyaluronic acid content was measured according to the manual in the kit using Hyaluronan ELISA kit (DHYAL0), and the hyaluronic acid content was calculated as Equation 5 below.

$$Hyaluronic acid content ( \% ) = ( Hyaluronic acid content of sample - treated group / hyaluronic acid content of sample - untreated group ) \times 100 ] \qquad [ Equation 5 ]$$

TABLE 9

| | | Hyaluronic |
|---|---|---|
| Sample | Treatment | acid content |
| (mixing ratio) | concentration | (%) |
| Example 1 (1:6) | 100 μg/mL | 89.94 ± 0.88 |
| Example 2 (1:4) | 100 μg/mL | 104.52 ± 0.53 |
| Example 3 (1:2) | 100 μg/mL | 106.07 ± 2.67 |
| Example 4 (1:1) | 100 μg/mL | 113.03 ± 1.26 |
| Example 5 (2:1) | 100 μg/mL | 119.11 ± 0.88 |
| Example 6 (4:1) | 100 μg/mL | 107.28 ± 1.85 |
| Example 7 (6:1) | 100 μg/mL | 94.44 ± 0.53 |
| Comparative | 100 μg/mL | 103.94 ± 2.89 |
| example 1 (1:0) | | |
| Comparative | 100 μg/mL | 105.03 ± 1.26 |
| example 2 (0:1) | | |

As a result, as shown in Table 9, it could be confirmed that the hyaluronic acid contents of 113.03% to 119.11% shown at the 100 μg/mL treatment concentration of Examples 4 to 5 were significantly increased, compared to the hyaluronic acid content of 103.94% to 105.03% shown at the 100 μg/mL treatment concentration of Comparative examples 1 to 2. It was confirmed that the skin moisturizing effect was excellent by confirming that the hyaluronic acid content of Example 5 was the highest among them.

So far, specific parts of the content of the present invention have been described in detail, so for those skilled in the art, it is clear that these specific descriptions are only preferable embodiments, and the scope of the present invention is not limited thereby. Accordingly, the substantial scope of the present invention will be defined by the appended claims and their equivalents.

1.-15. (canceled)

16. A method for anti-oxidation, wrinkle improvement, elasticity improvement and/or skin moisturizing, comprising:
administering a complex of an Indian gooseberry (Phyllanthus emblica) extract and a barley sprout (Hordeum vulgare) extract as an active ingredient to a subject in need thereof.

17. The method according to claim 16, wherein the Indian gooseberry extract or barley sprout extract is juiced or extracted with a solvent selected from the group consisting of water, low carbon alcohols having 1 to 4 carbon atoms and mixed solvents thereof.

18. The method according to claim 16, wherein the complex of the Indian gooseberry extract and barley sprout extract is obtained by juicing raw materials.

19. The method according to claim 16, wherein the Indian gooseberry extract or barley sprout extract is a dry powder.

20. The method according to claim 16, wherein the complex of the Indian gooseberry extract and barley sprout extract comprises the Indian gooseberry extract and the barley sprout extract at a weight ratio of 1:1 to 4:1 (Indian gooseberry extract:barley sprout extract).

21. The method according to claim 16, wherein the complex of the Indian gooseberry extract and barley sprout extract has DPPH scavenging activity, elastase inhibition activity, MMP-1 inhibition activity and a hyaluronic acid content increasing effect.

22. The method according to claim 16, wherein the complex of the Indian gooseberry extract and barley sprout extract is comprised in a health functional food product, and the method is carried out by administering the health functional food to the subject.

23. The method according to claim 16, wherein the complex of the Indian gooseberry extract and barley sprout extract is comprised in a food product, and the method is carried out by administering the health functional food to the subject.

24. A method for obtaining cosmetic effects selected from the consisting of anti-oxidation, wrinkle improvement, elasticity improvement and skin moisturizing, comprising:
administering a complex of an Indian gooseberry (Phyllanthus emblica) extract and a barley sprout (Hordeum vulgare) extract as an active ingredient to a subject in need thereof.

25. The method of according to claim 24, wherein the Indian gooseberry extract or barley sprout extract is juiced or extracted with a solvent selected from the group consisting of water, low carbon alcohols having 1 to 4 carbon atoms and mixed solvents thereof.

26. The method of according to claim 24, wherein the complex of the Indian gooseberry extract and barley sprout extract is obtained by juicing raw materials.

27. The method of according to claim 24, wherein the Indian gooseberry extract or barley sprout extract is a dry powder.

28. The method of according to claim 24, wherein the complex of the Indian gooseberry extract and barley sprout extract comprises the Indian gooseberry extract and the barley sprout extract at a weight ratio of 1:1 to 4:1 (Indian gooseberry extract:barley sprout extract).

29. The method of according to claim 24, wherein the complex of the Indian gooseberry extract and barley sprout extract has DPPH scavenging activity, elastase inhibition activity, MMP-1 inhibition activity and a hyaluronic acid content increasing effect.

30. The method according to claim 24, wherein the complex of the Indian gooseberry extract and barley sprout extract is comprised in a cosmetic product, and the method is carried out by applying the cosmetic product to the subject.
